(19) United States
(12) Patent Application Publication
(10) Pub. No.: US 2015/0359945 A1
(43) Pub. Date: Dec. 17, 2015

(54) ANTIMICROBIAL COATINGS

(71) Applicant: BOARD OF REGENTS, THE UNIVERSITY OF TEXAS SYSTEM, Austin, TX (US)

(72) Inventors: Joel ROSENBLATT, Pottstown, PA (US); Issam RAAD, Missouri City, TX (US)

(73) Assignee: BOARD OF REGENTS, THE UNIVERSITY OF TEXAS SYSTEM, Austin, TX (US)

(21) Appl. No.: 14/760,712

(22) PCT Filed: Jan. 9, 2014
(86) PCT No.: PCT/US2014/010897

§ 371 Date: Jul. 14, 2015

Related U.S. Application Data
(60) Provisional application No. 61/752,546, filed on Jan. 15, 2013.

(51) Int. Cl. A61L 31/10; A61L 31/14; A61L 31/16

(52) U.S. Cl. CPC A61L 31/10; A61L 31/16; A61L 31/145; A61L 2420/06; A61L 2420/02; A61L 2300/606; A61L 2300/45; A61L 2300/404; A61L 2300/206

(57) ABSTRACT

Provided are polymer-based surfaces (e.g., gloves) with an antimicrobial coating, e.g., comprising chlorhexidine and a partially hydrolyzed polyvinyl acetate copolymer, or a biguanide and a silicone. Methods of coating polymer-based surfaces with antimicrobial compositions are also provided.

This application claims the benefit of U.S. Provisional Patent Application No. 61/752,546, filed Jan. 15, 2013, the entirety of which is incorporated herein by reference.

### BACKGROUND OF THE INVENTION

1. Field of the Invention

The present invention relates generally to the field of infectious disease control. More particularly, it provides vinyl-based gloves coated with an antimicrobial composition.

2. Description of Related Art

The contamination of gloves with microorganisms is a major source of bacterial and fungal transmission in the medical and food industries. In the medical industry, latex gloves are worn during a medical procedure to provide a physical barrier between the patient's body or tissues and the hands, wrist and arm regions of a health care practitioner, such as a physician, nurse, phlebotomist, and the like. Rapid killing of microorganisms is very important because during momentary contacts or rapid manipulations on patients or equipment, microbes can be rapidly transferred by gloves from one surface to another. There is a need for medical devices and consumables (e.g., gloves or masks, etc.) with improved antimicrobial activity, and methods to impart antimicrobial activity on such devices, to prevent nosocomial infection.

### SUMMARY OF THE INVENTION

The present invention overcomes limitations in the prior art by providing new antimicrobial compositions and medical devices coated with the antimicrobial compositions. In some aspects, polymer gloves coated with the antimicrobial compositions are provided.

An aspect of the present invention relates to a medical device comprising a polymer-based surface, wherein an antimicrobial composition is present on at least a portion of the surface, and wherein the antimicrobial composition comprises: (a) a biguanide such as chlorhexidine, and a partially hydrolyzed polyvinyl acetate copolymer with a vinyl acetate content of from about 5% to about 95%; or (b) an aqueous or multiphase solution comprising a biguanide and a silicone; wherein if the silicone is a volatile silicone, then at least 0.01% of the volatile silicone can volatilize from the polymer based surface within 48 hours at about 50° C.; and wherein if the silicone is a nonvolatile silicone, then the ratio of biguanide/nonvolatile silicone is greater than 12. In some embodiments, the antimicrobial composition has been dried on the surface or the antimicrobial composition is a dry composition. In some embodiments, at least 0.01%, more preferably at least 0.05%, more preferably at least 0.1, more preferably at least 1%, at least 5%, at least 10%, at least 20%, at least 30%, at least 40%, at least 50%, at least 60%, at least 70%, at least 75%, at least 80%, even more preferably at least 90%, even more preferably at least 95%, even more preferably at least 99%, or up to at least about 99.99% of the volatile silicone can volatilize from the polymer based surface within 48 hours at about 50° C. or above; these percentages preferably refer to the percent of volatile silicone present in a wet antimicrobial composition, or in an antimicrobial composition on a surface prior to drying, that can volatilize from the polymer based surface. The partially hydrolyzed polyvinyl acetate copolymer may be a partially hydrolyzed vinyl alcohol vinyl acetate copolymer. The chlorhexidine may comprise greater than about 85% (w/w) or greater than about 90% (w/w) of the antimicrobial composition; these percentages preferably the percentage of chlorhexidine present in a antimicrobial composition that has been dried or has been subjected to drying. The medical device may be a glove. The glove may be a latex glove, a nitrile glove, a vinyl polymer glove, or a vinylidene polymer glove. The medical device may be a respiratory mask, a hair net or head cover, an exam table cover, an ear plug, a stethoscope cover, a footcover, a seatcover, a drape, a towel, a drain, a bag or pouch, a holder, a film, a tube, a gauze, a sponge, a mesh, a filter, a pad, a clip, a bandage, a drape, or an article of clothing. The chlorhexidine may be chlorhexidine gluconate. The antimicrobial composition may further comprise gentian violet or brilliant green. The partially hydrolyzed polyvinyl acetate copolymer may comprise from about 10% to about 90%, from about 10% to about 50%, from about 10% to about 25%, or from about 10% to about 15% vinyl acetate. The partially hydrolyzed polyvinyl acetate copolymer may comprise less than about 15% of the composition or from about 3% to about 5% of the composition; these percentages preferably refer to the percentage of partially hydrolyzed polyvinyl acetate copolymer present in a antimicrobial composition that has been dried or has been subjected to drying.

In some embodiments, the antimicrobial composition further comprises a gel-forming protein or gel-forming carbohydrate. The antimicrobial composition may further comprise a gel-forming protein, wherein the gel-forming protein is a gelatin, a collagen, a gelatin derivative, a collagen derivative, a starch, a chitosan, an alginate, or a gum. The antimicrobial composition may further comprise a gel-forming carbohydrate, wherein the gel-forming carbohydrate is a starch, chitosan, or alginate. The gel-forming protein or gel-forming carbohydrate may comprise from about 0.1% to about 5% of the composition or from about 1% to about 3% of the composition; these percentages preferably refer to the percentage of carbohydrate present in a antimicrobial composition that has been dried or has been subjected to drying. The medical device may be a glove, such as, e.g., a latex glove, a polyvinyl chloride glove, a nitrile glove, or a glove made of a laminate or blend of latex, polyvinylchloride, or nitrile. In some embodiments, the medical device is sterile. The antimicrobial composition may comprise an aqueous solution, suspension, or emulsion. In some embodiments, the antimicrobial composition comprises an aqueous or multiphase solution (e.g. a suspension or emulsion), comprising a biguanide and a silicone; wherein if the silicone is a volatile silicone, then at least 0.01% of the volatile silicone can volatilize from the polymer based surface within 48 hours at about 50° C.; and wherein if the silicone is a nonvolatile silicone, then the ratio of biguanide/nonvolatile silicone is greater than 12. The antimicrobial composition may comprises an aqueous or multiphase solution comprising a biguanide and a volatile silicone, wherein at least 0.01% of the volatile silicone can volatilize from the polymer based surface within 48 hours at about 50° C. The percentage of volatile silicone that can volatilize from the surface preferably refers to the percent of volatile silicone present in a wet antimicrobial composition, or in an antimicrobial composition prior to drying, that can volatilize from the polymer based surface. The antimicrobial composition may comprise a multiphase solution comprising a plurality of volatile phases that can concurrently volatilize (e.g., the volatile phases may at least 25% volatilize, at least 50% volailize, or at least 99% volatilize) from the polymer based surface within 48 hours at about 50° C. The antimicrobial composition may contain a volatile silicone and a humectant. The antimicrobial composition may comprise a solution or multiphase solution comprising a biguanide and a nonvolatile silicone, wherein the ratio of biguanide/nonvolatile silicone is greater than 12. In some embodiments, the biguanide concentration is greater than about 10%. The antimicrobial composition may further comprise a partially hydrolyzed polyvinylacetate. The partially hydrolyzed polyvinyl acetate copolymer may comprise from about 10% to about 50% or from about 10% to about 25% vinyl acetate; this percentage preferably refers to the percentage of vinyl acetate present in the copolymer. The partially hydrolyzed polyvinyl acetate copolymer may comprise less than about 15% or from about 3% to about 5% of the composition; these percentages preferably refer to the percentage of partially hydrolyzed polyvinyl acetate copolymer present in a antimicrobial composition that has been dried or has been subjected to drying The biguanide may be chlorhexidine, chlorhexidine gluconate, chlorhexidine salt, alexidine, octenidine, or polyhexamethyl biguanide. The volatile silicone may be a cyclomethicone such as, e.g., cyclotrisiloxane, cyclotetrasiloxane cyclopentasiloxane, cyclohexasiloxane, derivatives or mixtures thereof. The volatile silicone may be a dimethicone. The dimethicone may contain disiloxane, trisiloxane, tetrasiloxane, pentasiloxane, hexasiloxane, a polysiloxane or derivatives or mixtures thereof. In some embodiments, the volatile silicone comprises a mixture of a dimethicone (one or more dimehticones such as, e.g., PEG 8 dimethicone and cetyl PEG/PPG-10/1 dimethicone) and a cyclomethicone. The nonvolatile silicone may be a longer chain dimethicone having a viscosity of greater than 50 centistokes, greater than 100 centistokes, greater than 500 centistokes, greater than 1000 centistokes, greater than 5000 centistokes, greater than 10,00 centistokes, greater than 50,000 centistokes, or up to 100,000 centistokes, a dimethicone copolymer, a dimethicone copolyol, a dimethicone crosspolymer, a silicone glycol, a cetaryl dimethicone copolymer, a dimethiconol fluid, a PEG-dimethicone, a PPG-dimethicone, a PEG/PPG-dimethicone, a cetyl dimethicone, an alkyldimethicones, an alkyl silicone, a polyether silicone, a hydroxyl silicone, a polyether dimethicone, a silicone surfactants or emulsifier, an aminodimethicone, an amodimethicone, a cyclic siloxane, an alkoxylated dimethicone, a siloxane copolymer, or a siloxane copolymer derivative. The nonvolatile silicone may be a cyclic, grafted, or crosslinked siloxane. The antimicrobial composition may further comprise a hydrophobic oil or humectant, wherein the oil or humectant is pharmacologically acceptable for topical application to the skin. The oil may be coconut oil, an unsaturated alkanoic acid, an aloe oil, a tocopherol, a tocotrienol, or a dimethicone. The humectant may be glycerol, propylene glycol, polyethylene glycol, a polyethelene glycol polypropylene glycol copolymer, a polysorbate, a monogylyceride, diglyceride, triglyceride, fatty acid ether, glycerol or glycol conjugate. In some embodiments, the antimicrobial composition comprises cyclomethicone and PEG 8 dimethicone, PEG 33 dimethicone, and PEG 14. The antimicrobial composition may further comprise nonvolatile dimethicone. In some embodiments, the antimicrobial composition comprises gendine solution in a cyclomethicone emulsion. In some embodiments, the cyclomethicone emulsion comprises PEG 8 dimethicone and a d4 or d5 cyclomethicone. In some embodiments, the antimicrobial composition comprises PEG 8 dimethicone and Cetyl PEG/PPG-10/1 dimethicone.

Another aspect of the present invention relates to a method of imparting antimicrobial activity to a polymer-based surface, comprising contacting at least a portion of a polymer-based surface with the antimicrobial solution, and substantially drying the contacted surface; wherein the antimicrobial solution comprises: (a) chlorhexidine and a partially hydrolyzed polyvinyl acetate copolymer with a vinyl acetate content of greater than about 5%; (b) an aqueous solution or multiphase fluid comprising a biguanide and a silicone; wherein if the silicone is a volatile silicone, then at least 0.01% of the volatile silicone can volatilize from the polymer based surface within 48 hr at about 50° C.; and wherein if the silicone is a nonvolatile silicone, then the ratio of biguanide/nonvolatile silicone is greater than 12; or (c) applying an aqueous biguanide solution to the surface, and subsequently applying a silicone solution, e.g., preferably while the biguanide solution is drying, wherein if the silicone is a volatile silicone, then at least 0.01% of the volatile silicone can volatilize from the polymer based surface within 48 hr at about 50° C.; and wherein if the silicone is a nonvolatile silicone, then the ratio of biguanide/nonvolatile silicone is greater than 12. In some embodiments, the multiphase fluid comprises an aqueous fluid phase and a non-aqueous fluid phase. In some embodiments, at least 0.01%, more preferably at least 0.05%, more preferably at least 0.1%, more preferably at least 1%, at least 5%, at least 10%, at least 20%, at least 30%, at least 40%, at least 50%, at least 60%, at least 70%, at least 75%, at least 80%, even more preferably at least 90%, even more preferably at least 95%, even more preferably at least 99%, or up to at least about 99.99% of the volatile silicone can volatilize from the polymer based surface within 48 hours at about 50° C. or above; these percentages preferably refer to the percent of volatile silicone present in a wet antimicrobial composition, or in an antimicrobial composition on a surface prior to drying, that can volatilize from the polymer based surface. The partially hydrolyzed polyvinyl acetate copolymer may be a partially hydrolyzed vinyl alcohol vinyl acetate copolymer. The solution may further comprise a gel-forming protein or gel-forming carbohydrate. The solution may comprise gelatin. The solution may comprise greater than 85%, greater than 90%, or about 95% chlorhexidine. In some embodiments, the solution comprises about 95% chlorhexidine, about 3% partially hydrolyzed vinyl alcohol vinyl acetate copolymer, and about 2% gelatin; these percentages preferably refer to the percentage of the component present in a antimicrobial composition that has been dried or has been subjected to drying. The surface may comprise a nitrile, a latex, a polyvinyl chloride, a polyester, a polyamide, a polyurethane, a cellulosic, a silicone polymer, a fluoropolymer, or an olephin. In some embodiments, the surface is a vinyl-based surface. The surface may be comprised on a medical device such as, e.g., a respiratory mask, a hair net or head cover, an exam table cover, an ear plug, a stethoscope cover, a footcover, a seatcover, a drape, a towel, a drain, a bag or pouch, a holder, a film, a tube, a gauze, a sponge, a mesh, a filter, a pad, a clip, a bandage, a drape, or an article of clothing. The antimicrobial composition may be coated on a portion of the vinyl-based surface at a concentration effective to kill a bacterium or a fungus. The surface may be comprised on a glove such as, e.g., a latex glove, a polyvinyl chloride glove, or a nitrile glove. The vinyl-based surface may be contacted with a reactive gas, a gas-plasma, a reactive liquid, an interpenetrating network, or a hydrophilic coating, or may be exposed to radiation treatment. The contacting may comprise dipping or spraying. The method may further comprise using a roller process to apply the antimicrobial solution to the surface and dry the surface. In some embodiments, a heat tumble dryer is used to dry the antimicrobial solution on the surface. In some embodiments, the surface is comprised on a glove. In some embodiments, the antimicrobial composition is applied on the surface by immersing the surface in the antimicrobial composition and air drying.

In some embodiments, a silicone or an organo-silicone is included in the antimicrobial coating. For example, it is anticipated that a fluorosilicone, a volatile fluorocarbon, or a supercritical fluid such as supercritical carbon dioxide may be substituted for the silicone to achieve similar effect, e.g., to allow for application of the antimicrobial solution on a polymer based surface to result in an antimicrobial surface with reduced tackiness. In some embodiments, the fluorosilicone is a perfluoro-substituted dimethicone. In some embodiments, the volatile fluorocarbon is a hydrofluorocarbon or a chlorofluorocarbon.

Generally, the percentage of a component in an antimicrobial composition may refer to the percentage of the component in either a substantially, essentially, or completely dry antimicrobial composition, or in an antimicrobial composition that has been subjected to drying, unless it is stated otherwise. For example, an aqueous or multiphase solution present in an antimicrobial composition may refer to an antimicrobial composition that has not been subjected to drying or an antimicrobial solution that may be subsequently subjected to drying; thus, the percentage of a volatile silicone present in such an antimicrobial solution would generally refer to the percentage of volatile silicone in the solution, e.g., prior to drying the solution on a polymer-based surface such as the surface of a glove. As a result of the removal of the volatile silicone the percentages of the components will have changed relative to their composition in the resulting partially or completely dried antimicrobial composition.

As used herein, an “aqueous solution comprising a silicone” or “aqueous silicone solution” means a homogenous water-based solution containing a silicone, a heterogenous water-based solution containing a silicone solution, or a multiphasic solution comprising a water-based phase and a silicone phase. For example, the aqueous solution containing silicone may have two liquid phases (e.g., a water-based phase and a silicone phase). In some embodiments, a multiphase solution comprises at least two immiscible liquid phases (e.g., two immiscible volatile liquid phases such as a water-based phase and a silicone liquid phase). In some embodiments, a multiphase solution comprises three, four, or more phases.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Efficacy of antimicrobial Gendine-treated nitrile samples in eradicating E. coli and MRSA within 30 seconds and 1 minutes of contact.

FIGS. 2A-C: Efficacy of antimicrobial Gendine nitrile gloves tested for 1 minute exposure to multidrug resistant Gram-positive organisms (FIG. 2A), Gram-negative organisms (FIG. 2B), and Yeast (FIG. 2C).

### DESCRIPTION OF ILLUSTRATIVE EMBODIMENTS

The present invention provides, in various aspects, antimicrobial compositions that may be applied to a polymer-based surface of an article, such as a vinyl, PVC, or latex glove. The antimicrobial composition may comprise chlorhexidine and a partially hydrolyzed polyvinylacetate copolymer, such as a partially hydrolyzed vinyl alcohol vinyl acetate copolymer. The antimicrobial composition may be applied, e.g., to the outer surface of a medical or an industrial glove to minimize or reduce cross-contamination as a result of multiple contacts. As shown in the below examples, antimicrobial surfaces were able to eradicate over four logs of pathogenic microbes within about 30 seconds of contact. It has been observed that use of a partially hydrolyzed polyvinylacetate copolymer can allow for the use of less copolymer (e.g., less than about 5%) to effectively deliver higher concentrations of chlorhexidine (CHG) to a polymer surface, such as a glove, and the resulting surface was found to be less sticky or tacky as a result of the chlorhexidine. Thus, in some embodiments, a blocking agent is not required to reduce the stickiness or tackiness of a polymer surface, such as a glove surface.

In some embodiments, a gelatin-partially hydrolyzed polyvinyl acetate mixture can be used for binding CHG at very high concentrations (e.g., greater than 90% or about 95%) and facilitate film forming and coating adhesion properties. Part of the unexpected synergy in this combination is that in the dry state the CHG can act as both plasticizer of the polymers as well as antimicrobial agent. Upon moisture contact, as the CHG releases, the moisture uptaken can take over the plasticizing function to substantially reduce or prevent flaking or cracking of the coating.

In some embodiments, the antimicrobial surface may be coated with a composition comprising one or more antimicrobial agents in a single step. The polymer surface may be coated with an composition comprising: (a) chlorhexidine in an amount greater than about 80%, 81%, 81%, 83%, 84%, 85%, 86%, 87%, 88%, 89%, 90%, 91%, 92%, 93%, 94%, or 95% or any range derivable therein (b) a partially hydrolyzed polyvinyl acetate copolymer in an amount less than about 5%, such as about 3% of the total solids; and (c) optionally a gel-forming protein or carbohydrate such as gelatin in lesser quantity than the polyvinyl acetate polymer, preferably about 2% of the total solids. In certain aspects, the antimicrobial composition may also contain an additional antimicrobial agent, such as gentian violet.

### I. PARTIALLY HYDROLYZED POLYVINYL ACETATE COPOLYMERS

Some aspects of the present invention relate to the use of a partially hydrolyzed polyvinyl acetate copolymer to deliver one or more antimicrobial compounds to a polymeric surface. In various embodiments, it has been found that a partially hydrolyzed polyvinyl acetate copolymer can be used to effectively deliver higher concentrations of an antimicrobial composition, such as chlorhexidine, to a polymer surface, such as a glove, without adversely affecting the physical and mechanical properties of the resulting polymer surface. For example, it has been found that the tackiness or stickiness that can result from the deposition of chlorhexidine on a glove can be reduced by inclusion of a partially hydrolyzed polyvinyl acetate copolymer such as partially hydrolyzed vinyl alcohol vinyl acetate copolymer. Additionally, the use of a partially hydrolyzed vinyl acetate copolymer can allow for application of higher concentrations of antimicrobial agents, such as chlorhexidine. Since less total solution may be needed to apply a given concentration of an antimicrobial composition, higher concentrations of antimicrobial compounds can be applied to a surface, such as the exterior of a glove, or cost savings may be achieved by using a reduced total volume of solution for application to a glove. In some embodiments, the polymer is not a vinylpyrrolidone-vinyl acetate copolymer. In some embodiments, the partially hydrolyzed polyvinyl acetate copolymer is a partially hydrolyzed vinyl alcohol vinyl acetate copolymer.

Partially hydrolyzed polyvinyl acetate polymers differ significantly from substantially fully or completely hydrolyzed polyvinyl acetate polymers. Polyvinyl acetate can be partially or completely hydrolyzed to give polyvinyl alcohol. Polyvinyl acetate is a polymer having a hydrophobic backbone chain but a moderately hydrophilic side chain due to the presence of the acetate group. The hydrophilicity can be increased via transformation of the acetate group into an alcohol. Vinyl acetate-vinyl alcohol copolymers are typically industrially prepared starting from polyvinyl acetate, in which the acetate groups are partially substituted by hydroxyl groups, both by direct hydrolysis and by transesterification. The parameters characterizing the copolymer and determining its properties are: the proportion between the number of hydroxyl groups and the total number of groups (acetate+hydroxyl) present in the copolymer, also known as degree of hydrolysis of the copolymer, and the average molecular weight of the copolymer, which depends on the molecular weight of the starting polyvinyl acetate and on the hydrolysis degree. Because of the differences in the ratio of acetate and hydroxyl groups and their different hydrophobicities/hydrophilicities the physical-chemical properties of solutions of fully and partially hydrolyzed polyvinylacetate can be different. These include water binding and binding to other solutes or to hydrophobic surfaces.

For example, the following method may be used to generate a 10% (w/w) partially hydrolyzed polyvinyl acetate (PVA) solution in water. 10 grams of 11-13% partially hydrolyzed polyvinyl acetate (vinyl alcohol content 87-89%) can be dissolved in 90 grams water. The solution may then be heated and stirred, e.g., for about 4 hours, to facilitate polymer swelling and dissolution.

The partially hydrolyzed polyvinyl acetate copolymer can contain about 5%-95% vinyl acetate. The partially hydrolyzed polyvinyl acetate copolymer preferably contains at least about 5%, 6%, 7%, 8%, 9%, 10%, 11%, 12%, 13%, 14%, 15%, or about 5-95%, 5-90%, 5-50%, 5-25%, 10-25%, or 10-15%, or any range derivable therein, of vinyl acetate. The polyvinyl acetate copolymer may be a vinyl alcohol-vinyl acetate copolymer wherein the amount of vinyl acetate copolymer exceeds the amount of other monomers. The amount of copolymer in a coating may be less than about 15%, 14%, 13%, 12%, 11%, 10%, 9%, 8%, 7%, 6%, 5%, or less than about 4% of the total solids. In some embodiments, the vinyl acetate comprises about 2%, 3%, 4%, of 5%, or any range derivable therein, of the total solids. Other polymers that may be substituted for or used in combination with a partially hydrolyzed polyvinyl acetate copolymer include copolymers resulting from copolymerization of vinyl acetate with other monomers, e.g., where the same or a similar proportion of the vinyl acetate groups in the final copolymer are hydrolyzed to vinyl alcohol. This also applies to block and graft copolymers of vinyl acetate. Other monomers that can be copolymerized with vinyl acetate include vinyl pyrolidones, ethylene vinyl acetates, vinyl phthalates, vinyl chlorides and methacrylates.

### II. ANTIMICROBIAL AGENTS

The term “antimicrobial composition,” as used herein, refers to a composition that comprises an agent, such as an antiseptic, capable of preventing or reducing the growth or reproduction of a microorganism, such as a bacterial or fungal microorganism, or of killing a microorganism. Antiseptics that may be included in an antimicrobial composition of the present invention include, e.g., alexidines, octenidines, polyhexamethylene biguanide, gentian violet, and/or brilliant green.

The term “antiseptic” as used herein refers to a compound or agent that is capable of preventing or reducing the growth or reproduction of a microorganism (such as bacteria, fungi, protozoa, and viruses), or of killing a microorganism, but which is generally not applied in the treatment of a systemic infection in a subject, usually because of limitations related to absorption, penetration, or systemic toxicity. A non-limiting class of antiseptics that may be included in an antimicrobial composition of the present invention includes guanidium compounds, such as chlorhexidine. Other examples of antiseptics that may be used include phenoxide antiseptics (e.g., clofoctol, chloroxylenol, triclosan), quaternary ammonium compounds, cetyl pyridinium compounds, iodine compounds, hypochlorites, menthols, eucalyptols, thymols, salicylates, chlorxylenols, aldehydes, glutaraldehyde, peptides, peptide mimetics, dyes, acids, bases, oxidizers, gardine, gendine, genlenol, genlosan, or genfoctol.

The term “bacterial and fungal organisms” as used in the present invention means all genuses and species of bacteria and fungi, including but not limited to all spherical, rod-shaped, and spiral bacteria. Non-limiting examples of bacteria include staphylococci (e.g., Staphylococcus epidermidis, Staphylococcus aureus), Enterrococcus faecalis, Pseudomonas aeruginosa, Escherichia coli, Acinetobacter baumanni, Klebsiella pneumoniae, among other gram-positive bacteria and gram-negative bacilli. Some bacteria may have antibiotic resistance properties, such as methicillin-resistant S. aureus, and vancomycin-resistant Enterococci. Non-limiting examples of fungal organisms include Candida albicans, Candida glabrata, and Candida krusei.

a. Chlorhexidine

Chlorhexidine is an antiseptic cleansing agent that is active against staphylococci and other gram-positive bacteria, as well as against various fungi. Chlorhexidine may be included in an antiseptic solution described herein. Chlorhexidine is soluble in both water and organic solutions, including alcohols, ketones, ethers, aldehydes, acetonitrile, acetic acid, methylene chloride, and chloroform. As used herein, the term “chlorhexidine” includes salts of chlorhexidine, such as the diacetate, dihydrochloride, diphosphanilate, and digluconate salts. A preferred chlorhexidine salt that may be used with the present invention is chlorhexidine digluconate, i.e., chlorhexidine gluconate (CHG).

In various embodiments, CHG may be present in an antimicrobial composition that is applied to a polymer surface, such as a polymer glove, in an amount greater than about or about 80%, 85%, 86%, 87%, 88%, 89%, 90%, 91%, 92%, 93%, 94%, 95%, 96%, 97%, or any range derivable therein, by weight of total solids. In some embodiments, CHG may be present in the antimicrobial composition in an amount from greater than about 85% to about 96%, from greater than about 85% to about 95%, from greater than about 90% to about 95%, or about 95% by weight of total solids. As shown in the below examples, the use of a partially hydrolyzed polyvinylacetate copolymer, such as partially hydrolyzed vinyl alcohol vinyl acetate copolymer, can allow in various embodiments for the inclusion of higher concentrations of CHG to be effectively applied to a polymer surface, such as a polymer glove.

Without wishing to be bound by any theory, CHG can act as a plasticizer when applied to the surface of a polymer glove. In various aspects, it has been discovered a gelatin-partially hydrolyzed polyvinyl acetate mixture can allow for binding CHG at very high CHG levels and facilitate film forming and coating adhesion properties. Part of the unexpected synergy in this combination is that in the dry state the CHG acts as both plasticizer of the polymers as well as antimicrobial agent. Upon moisture contact, as the CHG releases, the moisture uptaken can take over the plasticizing function to substantially minimize or prevent flaking or cracking of the coating. Generally, the surface to which the film adheres can be wetted or solvated by water or volatile solvents in which the coating composition is dissolved and applied. Surface chemistries can be modified to render them wettable or solvatable by a variety of preparations including gas plasma treatment, irradiation or surface chemical modification. Texturing or microtexturing of surfaces can also enhance wettability.

b. Gentian violet

Gentian violet (also known as Crystal violet, Methyl Violet 10B, hexamethyl pararosaniline chloride, or pyoctanin(e)) is a triarylmethane dye that can be included in an antiseptic composition or coating of the present invention. This dye has been used as a histological stain and in Gram's method of classifying bacteria. Gentian violet has antibacterial, antifungal, and anthelmintic properties. In some embodiments, Gentian violet is included in an antiseptic composition that will be coated onto a polymer surface, such as a glove, in an amount of, e.g., about 1 part per million (ppm) to 100,000 ppm, 1-10,000 ppm, 1-1000 ppm, or about 1-10 ppm.

Gendine comprises a composition of the combination of gentian violet (GV) and chlorhexidine (CHG). Gendine may be included in an antiseptic composition that will be used to coat a polymer surface comprising CHG in an amount of about 1 ppm-500,000 ppm and GV in an amount of about 1 ppm-100,000 ppm. In some embodiments, about 20% (about 200,000 ppm) of CHG and about 1-10 ppm GV may be included in an antiseptic composition for coating a polymer surface.

Gendine may be particularly useful for impregnating various device polymers, such as polyvinyl chloride and polyurethane polymers. Gentian violet alone can be used to impregnate the surfaces of various polymers, including polyvinylchloride. However, gentian violet alone may have limited or no activity against Pseudomonas aeruginosa, which can cause nosocomial pneumonia and nosocomial urinary tract infections. Antiseptics, such as chlorhexidine, generally cannot easily attach on their own onto polymer surfaces, such as polyvinyl chloride. They typically require an impregnating vehicle. Furthermore, on their own they may exhibit limited activity against Pseudomonas aeruginosa. On the other hand, upon the binding of gentian violet with chlorhexidine, the antiseptic agent synthesized, may be used as a more potent and effective broad-spectrum antiseptic and may more easily be used to coat or impregnate various device or polymer surfaces.

c. Brilliant green

Brilliant green is also a triarylmethane dye that may be included in antiseptic solutions and coatings of the present invention. Brilliant green is also referred to as Malachite green G, Emerald green, Solid green JJO, Diamond green G, Aniline green, Benzaldehyde green, or Fast green J. Brilliant Green can effectively kill gram-positive bacteria. Brilliant Green may result in little or no irritation of mucous membranes as compared to other antiseptics such as iodine. Brilliant green may be included in an antiseptic composition that will be coated onto a polymer surface, such as a glove, in an amount of, e.g., about 1-100000 ppm, 1-10000 ppm, 1-1000 ppm, or about 1-10 ppm.

Gardine comprises a composition of the combination of brilliant green and chlorhexidine and may be coated on a polymer surface in embodiments of the present invention. Gardine may be included in an antiseptic composition that will be used to coat a polymer surface in an amount of, e.g., about 1-100000 ppm, 1-10000 ppm, 1-1000 ppm, or about 1-10 ppm.

d. Other

One or more additional antiseptic or antimicrobial compounds may be included in an antimicrobial coating, for example on a glove, of the present invention. For example, biguanide is an antimicrobial agent that may be included in an antimicrobial coating, e.g., in combination with CHG. Biguanide may be included in an antimicrobial solution to be coated on a polymer surface, e.g., in an amount of about 1-500,000 ppm in the coating solution. In some embodiments, a biguanide is included in an antimicrobial coating in combination with gentian violet and/or brilliant green. For example, at least about 1 part per million of each of biguanine and gentian violet may be used. In some embodiments, a biguanide-brilliant green mixture with at least 1 part per million of each agent may be included in an antimicrobial coating.

Other antiseptics or detergents may be included in an antimicrobial coating or an antimicrobial solution that may be applied to a polymer surface include antimicrobial peptides, chelators, and/or other membrane disrupting molecules. Suitable components for inclusion in an antimicrobial solution for application to a polymer surface may be found, e.g., in U.S. Pat. No. 8,137,735, U.S. Pat. No. 5,357,636, US2008/0081020, US2010/0233223, U.S. Pat. No. 5,438,709, U.S. Pat. No. 4,853,978, or US 2008/0183152, which are incorporated herein in their entirety without disclaimer.

### III. THICKENERS OR GEL-FORMING AGENTS

In some embodiments, a gel-forming protein or gel-forming carbohydrate may be included as a thickener in antimicrobial coating or solution. The inclusion of a thickener or gel-forming agent may aid in application of the antimicrobial solution to a polymer surface. In various embodiments, the gel-forming protein or gel forming carbohydrate may comprise about 1%, 2%, 3%, 4%, or about 5%, or any range derivable therein, of the antimicrobial composition that is applied to a polymer surface, such as a polymer glove.

In some embodiments a gel-forming protein may be used. Examples of proteins having gel-forming properties include, e.g., gluten, collagen, gelatin, egg albumen, soy bean protein, pectins, and mucins, or a derivative thereof. In some embodiments, a mixture of proteins, or a mixture of proteins and carbohydrates may be used as a thickener or gel forming agent.

In some embodiments a gel-forming carbohydrate may be used. Examples of carbohydrates having gel-forming properties include, e.g., chitosan, alginate, and starch, such as tapioca starch, wheat starch, potato starch, corn starch, and gums such as guar, agarose, rhamsan, gellan, glucan, galactomanan, Welan, Xanthan or acacia, or a derivative thereof. In some embodiments, a mixture of carbohydrates may be used as a thickener or gel forming agent.

In some embodiments, the gel forming protein is a gelatin or collagen derivative. Examples of gel forming carbohydrate that may be used include, e.g., a starch, chitosan, alginate, and gums, or a derivative thereof. If a gel-forming protein and/or carbohydrate is included in an antimicrobial coating, it may be included in lesser quantity than a partially hydrolyzed polyvinyl acetate polymer. For example, a gel-forming protein or carbohydrate may be included in an antimicrobial coating in an about of about 1-3% or about 2% of the total solids.

### IV. SOLUTIONS

In some aspects, an antimicrobial solution may be used to coat a polymer surface, such as a glove surface. A portion of the glove surface may be coated with the solution by dipping the article or glove into the solution or spraying the solution onto the glove. Generally, surface must first be wettable by the solution being applied. For example, surfaces can be rendered wettable or solvatable by reactive gas, radiation, chemical treatment, and/or texturing. In some embodiments, the solution is a water-based solution. Nonetheless, it is anticipated that other solutions, including ethanol, isopropanol, or a volatile organic solvent such as, e.g., methanol may be used to coat or deliver an antimicrobial composition to a surface or glove. As used herein, the term “solution” refers to a composition that is substantially liquid in form; the solution may have a gel-like consistency due to the inclusion of a thickener or gelling agent. Generally, an antimicrobial solution applied to a polymer surface may be allowed to dry before use.

### V. Polymer-Based Surfaces and Articles

Aspects of the present invention relate to polymer-based surfaces and articles, such as gloves, that have been coated with an antimicrobial composition. In some aspects, it has been found that use of a partially hydrolyzed polyvinyl acetate copolymer may be particularly useful for applying antimicrobial compositions, e.g., containing chlorhexidine, to a polymer surface, such as a vinyl-based surface. Polymer surfaces that may be used include nitrile (nitrile butadiene rubber), latex rubbers (polyisoprene and copolymers or derivatives), styrene-butadiene, acrylonitrile, butadiene, styrene, acrylic polymers, vinyl acetate polymers, acrylic latex polymers, polyvinylchloride, polyester, polyamide, polyurethane, polychloroprene (neoprene), vinyl acrylics, polyvinylacetate (PVA), cellulosics, olephins, spun bonded olephins, silicones, Tyveks, and fluoropolymers. In some embodiments, the surface may be neoprene (chloroprene), a butyl rubber, a chlorobutyl rubber, or a bromobutyl rubber.

In some embodiments, the polymer is a vinyl-based polymer. Vinyl groups in monomers can polymerize with the aid of a radical initiator or a catalyst to form a vinyl polymers. In these polymers, the double bond in each vinyl monomers can react to form monomers joined by single bonds. Vinyl groups do not exist in vinyl polymer; the term “vinyl polymer” generally refers to the precursor used to generate the polymer. It is sometimes important to ascertain the absence of unreacted vinyl monomer in the final product when the monomer is toxic or reduces the performance of a plastic. Some examples of vinyl and vinylidene polymers that may be coated with an antimicrobial composition according to the present invention include, e.g., polyvinyl chloride (PVC), Polyvinyl fluoride (PVF), Polyvinyl acetate (PVAc), Polyvinylidene chloride (PVDC), Polyvinylidene fluoride (PVDF), and Fluorinated propylenes and ethylenes and copolymers.

Personal protection products, such as gloves, masks, nets, covers, plugs, medical devices, holders, films, tubes, drains, gauzes, sponges, meshes, filters, pads, footware, a clip, a thread, a staple, a bandage, covering, clothing, towel, drapes, diapers, bags, pouches may be treated coated with an antimicrobial composition as described herein. In some embodiments, the glove is a food-contact glove, a dental glove, an industrial glove, a laboratory glove, a medical exam glove, or a surgical glove. In is envisioned that any personal protection or comfort items whose surface is rendered wettable by the coating composition by either chemical modification or microsurface texturing. In addition to coating the devices themselves an antimicrobial coating composition of the present invention may be applied to packaging, such as medical device packaging, in order to maintain sterility.

The terms “antimicrobial medical device” and “medical device” as used herein, refer to an instrument, apparatus, implement, machine, contrivance, implant, or other similar or related article, including a component part, or accessory, which is subjected to sequential antimicrobial contact as described, and is intended for use in the diagnosis, treatment, and/or prevention of disease or other health-related condition in a subject. In some embodiments, the medical device is a personal protection device. The subject can be any vertebrate, such as a mammal or a human. Non-limiting examples of antimicrobial medical devices include vascular catheters, such as peripherally insertable central venous catheters, dialysis catheters, long term tunneled central venous catheters, peripheral venous catheters, single-lumen and multiple-lumen short-term central venous catheters, arterial catheters, pulmonary artery Swan-Ganz catheters, and the like; urinary catheters, other long term urinary devices, tissue bonding urinary devices, renal stents, penile prostheses, vascular grafts, vascular access ports, wound drain tubes, hydrocephalus shunts, ventricular drainage catheters, neurologic and epidural catheters, peritoneal dialysis catheters, pacemaker capsules, artificial urinary sphincters, small or temporary joint replacements, dilators, heart valves, orthopedic prosthesis, spinal hardware, surgical site repair mesh (e.g., hernia mesh), endotracheal tubes, biliary stents, gastrointestinal tubes, gloves (including latex, non-latex, and nitrile), other medical garb, charts, bed rails, condoms, colorectal tract implants, male and female reproductive implants, cosmetic or reconstructive implants (e.g., breast, chin, cheek, buttock, nasal), medical device envelopes and pouches, including stethoscope drums, orthopedic implants (e.g., for a joint, such as a knee, hip, elbow, shoulder, or ankle), prostheses, external fixation pins, intramedullary rods and nails, spine implants), other medical and indwelling devices that may be subject to microbial infestation and/or activity. The medical device may be a condom, cervical cap, or a disposable contraceptive device. In some embodiments, antimicrobial compositions or solutions of the present invention may be used to substantially disinfect or reduce the growth of a microorganism (e.g., a bacteria or fungi) on an exterior surface of a glove. The medical device may be a respiratory mask, a hair net or head cover, an exam table cover, an ear plug, a stethoscope cover, a toilet or other seat cover, shoe cover, flip-flop or other footware, shoe pad, sock, finger cot, nose plug, drape, glasses or eye shields.

In some embodiments, at least a portion of an exterior or outer surface of a medical device or glove is coated with an antimicrobial composition as described herein. The outer surface refers to a surface that may come in contact with other objects, such as patients, medical instruments, tabletops, or counters. The outside surface may be present on a glove or medical device that may come into contact with a patient or may be exposed to possible contamination. In some embodiments, both the exterior and the interior of a glove may be coated with the antimicrobial composition. In some embodiments, the entire exterior and/or interior surface of a glove or other medical device is coated with the antimicrobial composition.

Medical devices that are amenable to treatment according to the method of the present invention generally include medical devices that comprise a non-metallic surface, such as a surface comprising rubber, plastic, polyethylene, polyurethane, silicone, polytetrafluoroethylene (PTFE), polyethylene terephthalate (PET), latex, nitrile, cellulosic, vinyl, and other polymeric and elastomeric materials. Generally, a silicone surface should be made wettable prior to applying an antimicrobial composition of the present invention. Those skilled in the art will appreciate that the listing of materials as described herein is exemplary only, and is not intended to be exclusive. Other materials that are amenable to treatment as described herein are also within the scope of the present invention. Generally, a surface on the medical device it must be made wettable so a film forms that an antimicrobial solution can adhere to the surface.

### VI. METHODS OF PRODUCING ANTIMICROBIAL POLYMER-BASED SURFACES AND GLOVES

Methods of producing polymeric gloves and applying antimicrobial solutions to gloves are known. The below methods are provided as illustrative examples only, and it is anticipated that other methods may be used with the present invention.

b. Wettability

The surface of the glove can be made wettable by contact with reactive gas, gas-plasma, reactive liquid, interpenetrating network, hydrophilic coating, radiation treatment, and/or texturing or microtexturing. Methods for making a surface wettable are known in the art. For example, methods that may be used with the present invention to make a surface wettable include, for example, the use of reactive gas (e.g., Allmear et al., 1988), reactive liquid (e.g., Uyama et al., 1998; U.S. Pat. No. 5,364,918), gas plasma (e.g., Hegemann et al. 2003; Weikart et al., 2000), interpenetrating network (e.g., U.S. Pat. No. 6,120,904), hydrophilic coating (e.g., U.S. Pat. No. 4,729,914; U.S. Pat. No. 7,553,511), radiation treatment (e.g., Ferreira et al., 2006; Ma et al. 2002), texturing and microtexturing (e.g., Bico et al., 2002), composites (e.g., Fotiadou et al., 2010), adsorption (e.g., Coupe et al., 2001). Chlorination and/or polyurethane treatments are examples of finishes that may be used to improve the wettability of gloves.

The terms “reactive gas” and “gas-plasma,” as used herein, refer to a highly reactive gas containing free electrons, positive ions, and other chemical species. The term “reactive liquid,” as used herein, refers to a condensed vapor or solution containing chemically reactive molecules that can react with a surface and alter the chemical properties of the surface. The term “interpenetrating network,” as used herein, can be defined broadly as an intimate network of two or more polymers at least one of which is either synthesized or cross-linked in the presence of the other. The term “hydrophilic coating,” as used herein, refers to a surface that upon contact with an aqueous liquid spontaneously forms an aqueous film covering or absorbs the aqueous liquid. The term “radiation treatment,” as used herein, can mean gamma radiation from a suitable source.

c. Coating

The antimicrobial composition used in the method of the present invention can be applied to a glove surface using conventional equipment and techniques readily available to those in the field of manufacturing objects with polymer-based surfaces, such as dipping, spraying, tumbling, or any suitable method for forming a conformal wet film on the polymer surface. Examples of coating techniques are described in U.S. Pat. Pub. No. 2004/0126604 and U.S. Pat. Pub. No. 2004/0241201. For example, when preparing surgical gloves, spraying may be used to apply an antimicrobial composition to the surface of the glove. For the preparation of examination gloves, dip coating may be used for application of an antimicrobial solution. After coating, the article may then be dried.

Emollients, surfactants, humectants, or tactile agents can be added to the coating composition to improve the feel on the skin and the handling properties or extensional handling properties of the coated surface. Silicone emulsions are examples of tactile agents that impart a smooth and slippery feel on skin. Scented, coloring, texturing, indicator, and/or flavoring agents with some water solubility may also be included in the coating. Volatile fluids can also help to improve processing as well as the finish of dried coated articles. Volatile fluids that may be used include volatile silicones such as cyclomethicones, dimethicones and mixed silicone fluids; in some embodiments, the volatile liquid, volatile silicone, or mixed silicone fluid has at least 0.01 wt %, 0.02 wt %, 0.03 wt %, 0.04 wt %, 0.05 wt %, 0.06 wt %, 0.07 wt %, 0.08 wt %, 0.09 wt %, 0.1 wt %, 0.2 wt %, 0.3 wt %, 0.4 wt %, 0.5 wt %, 1 wt %, 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, 90 wt %, 95 wt %, 99 wt %, 99.5 wt %, 99.9 wt %, or up to 99.99% capable of evaporating over 48 hours at 50° C. or above. In some embodiments, a volatile solid such as, e.g., hexamethyl cyclotrisiloxane, dichlorobenzene, or a napthalene solid may be used as a processing aid or included in an antimicrobial coating of the present invention. Silicones and other volatiles can also dehydrate the coating on evaporation such that it begins to lose the ability to stretch with the underlying elastomeric glove. Glycerol, propylene glycol, polyethylene glycol, polyethelene glycol polypropylene glycol copolymers, polysorbates, monogylycerides, diglycerides, triglycerides, fatty acid ethers, glycerol or glycol conjugates are examples of humectants that may be used to restore extensibility to excessively dehydrated coatings.

d. Roller Process for Coating and Drying Gloves:

In some embodiments, a roller process may be used for coating and/or drying gloves of the present invention. In some embodiments, the following method is used. Individual gloves are may be laid flat on an open mesh conveyer belt. The conveyor may feed the gloves through a double roller with both rollers wetted with coating solution. The rollers may gently press on the gloves and apply coating solution to all or substantially all of the exterior surfaces. A second set of rollers (non-wetted) may be used to wipe away excess solution. The conveyer may continue through a pass-through radiant or convection oven. The oven may dry the coating on the gloves. The gloves may then fall into a collection bin as the conveyor belt returns to the beginning.

Coating and drying may be performed, e.g., as described in U.S. Pat. No. 6,630,152. For example, a coating fluid may be sprayed or misted to wet the gloves, followed by tumble drying under heat to evaporate excess liquid and dry the coating on the glove surfaces. Coating of the liquid may also be performed, e.g., as described in US20110099688; for example, a coating liquid may be applied to gloves in a washer, followed by tumbling of the gloves to promote even coating, and followed by tumble drying in a heat tumble dryer such as a cyclone tumble dryer.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Optimization of Gendine-Based Coating Solution

Preparation of Glove Coating Solutions:

A 10% (w/w) partially hydrolyzed polyvinyl acetate (PVA) solution in water was made by dissolving 10 g of 11%-13% partially hydrolyzed polyvinyl acetate (vinyl alcohol content 87%-89%) in 90 g water. The solution was heated and stirred for four hours to facilitate polymer swelling and dissolution. A 20% gelatin solution was made by dissolving 20 g porcine gelatin in 80 g of hot water. A 0.1% Gentian violet (GV) solution was made by dissolving 100 mg of Gentian violet in 99.9 g water. A 20% chlorhexidine gluconate (CHG) solution in water was used as supplied from the manufacturer. Solution Gendine A consisted of 24 g CHG solution+6 g PVA solution+2 g gelatin solution+0.05 g GV solution in a 50 mL vial (approximately 83% CHG content in the coating solids).

Glove Coating Method:

Nitrile glove substrates were fingers cut off from commercial nitrile exam gloves taken directly from the box. The fingers were placed over the rounded ends of glass test tubes, slightly stretched to remove any creases or wrinkles and then clamped. The clamped samples were dipped into the vials for a few seconds then retracted. Excess solution was allowed to drip off of the nitrile fingers and then the coated fingers were allowed to dry. A shiny lustrous film formed on the nitrile surface upon drying.

Mechanical Testing:

The coated nitrile fingers were repeatedly stretched to 150% of their original length to attempt to delaminate or fracture the coating. The coatings did not delaminate after 10 such stretches in either wet or dry states.

Antimicrobial Efficacy Testing:

The coated fingers were tested per ISO 22196 (International Standards Organization method 22196). Briefly, the coated fingers were removed from the test tubes, the rounded tips cut off, and the resulting cylinders slit lengthwise. Resulting rectangular films were secured over the opening of a 50 mL tube (coated side pointing up) to ensure that the surface of the tested films were flat. One hundred microliters of bacterial inoculum, clinical isolates of methicillin-resistant Staphylococcus aureus (MRSA) or Escherichia coli (E. coli) were pipetted onto the surface of film. Inoculum concentrations of approximately 5×10⁵ cfu/mL were used. A square microscope coverslip (22 mm per side) was placed on top of the bacterial inoculum to spread a thin film of inoculum. The inoculum was exposed for either 30 seconds or 1 minute in this manner. Testing was performed in duplicate for each organism or exposure time. After exposure, antimicrobial activity was halted by submersion and stirring in 10 mL of Dey-Engle Neutralizing Broth. One hundred microliters of the resulting broth was aspirated and either serially diluted or directly plated onto agar plates. The plates were incubated 24 hours at 37° C. and the number of colonies formed was counted. The number of colonies on the film was calculated by multiplying the number of colonies that were counted times 1,000 and the serial dilution factor (if not 1). An uncoated glove finger was used as a positive control. The log reduction was computed as the difference in the log of the number of colonies on the control finger from the coated finger. If no colonies were counted from a film then, for purposes of computing a log reduction, the log was assigned a value of zero.

Results:

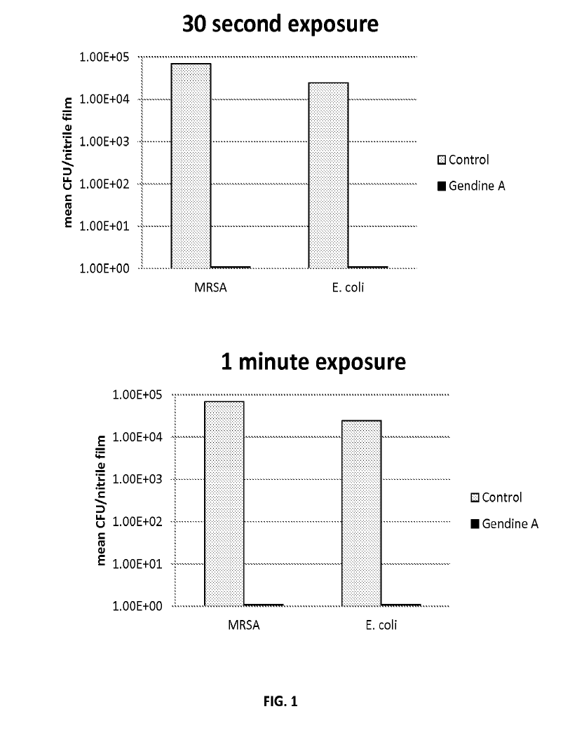

The Gendine-treated nitrile samples completely eradicated over 4 logs of MRSA and E. coli within 30 seconds of contact (FIG. 1; Table 1).

TABLE 1
Antimicrobial efficacy testing against
E. coli and MRSA bacterial challenges.

| | Exposure | | | Mean CFU | Log |
|---|---|---|---|---|---|
| Coating | time | | Organism | recovered per film | reduction |
| Control | 30 | sec | MRSA | 5 × 10⁴ | — |
| Control | 30 | sec | E. coli | 2 × 10⁴ | — |
| Gendine | 30 | sec | MRSA | 0 | 4.7 |
| Gendine | 30 | sec | E. coli | 0 | 4.3 |
| Control | 1 | minute | MRSA | 5 × 10⁴ | — |
| Control | 1 | minute | E. coli | 2 × 10⁴ | — |
| Gendine | 1 | minute | MRSA | 0 | 4.7 |
| Gendine | 1 | minute | E. coli | 0 | 4.3 |

The test above was repeated with Pseudomonas aeruginosa substituted for E. coli (Table 2).

TABLE 2
Antimicrobial efficacy testing against Pseudomonas
aeruginosa (PA) and MRSA bacterial challenges.

| | Exposure | | | Mean CFU | Log |
|---|---|---|---|---|---|
| Coating | time | | Organism | recovered per film | reduction |
| Control | 30 | sec | MRSA | 1.3 × 10⁴ | — |
| Control | 30 | sec | PA | 2.5 × 10⁴ | — |
| Gendine | 30 | sec | MRSA | 0 | 4.1 |
| Gendine | 30 | sec | PA | 0 | 4.4 |
| Control | 1 | minute | MRSA | 1.3 × 10⁴ | — |
| Control | 1 | minute | PA | 2.5 × 10⁴ | — |
| Gendine | 1 | minute | MRSA | 0 | 4.1 |
| Gendine | 1 | minute | PA | 0 | 4.4 |

To prepare glove coating solutions with higher CHG content in coating solids, component solutions were combined according to the proportions in Table 3. The 20% chlorhexidine gluconate (CHG), 10% partially hydrolyzed polyvinyl acetate (PHPA), 20% gelatin, and 0.1% Gentian violet (GV) component solutions were prepared as described above.

TABLE 3
Proportions of component solution used
to make the coating stock solutions.

| | Gendine B | Gendine C | Gendine D |
|---|---|---|---|
| Mass (g) 20% CHG solution | 24.0 | 27.0 | 28.0 |
| Mass (g) 10% PHPA solution | 4.0 | 3.0 | 2.0 |
| Mass (g) 20% Gelatin solution | 1.3 | 1.0 | 0.7 |
| Mass (g) 0.1% GV solution | 0.05 | 0.05 | 0.05 |
| % CHG in coating solids | 87% | 91% | 94% |

Note that the percent of CHG in coating solids in Gendine A was approximately 83%.

Mechanical testing was performed as described above. The coated nitrile fingers were repeatedly stretched to 150% of their original length to attempt to delaminate or fracture the coating. The coatings did not delaminate after 10 such stretches in either wet or dry states.

Antimicrobial testing was performed per ISO 22196 as described above with 45 second exposures to inocula prior to neutralization. The results are tabulated below in Table 4.

TABLE 4
Antimicrobial testing of coating
solutions with higher CHG content.

| | Exposure | | Mean CFU | Log |
|---|---|---|---|---|
| Coating | time | Organism | recovered per film | reduction |
| Control | 45 sec | MRSA | 1.2 × 10⁵ | — |
| Control | 45 sec | E. coli | 4 × 10⁴ | — |
| Gendine B | 45 sec | MRSA | 0 | 5.1 |
| Gendine B | 45 sec | E. coli | 0 | 4.6 |
| Gendine C | 45 sec | MRSA | 0 | 5.1 |
| Gendine C | 45 sec | E. coli | 0 | 4.6 |
| Gendine D | 45 sec | MRSA | 0 | 5.1 |
| Gendine D | 45 sec | E. coli | 0 | 4.6 |

### Example 2

### Antimicrobial Efficacy of Gendine Medline Nitrile Gloves

The inventors developed and tested Gendine-coated nitrile coated glove fingers for efficacy by the International Standard ISO 22196.

Materials:

Medline nitrile glove fingers were coated with a single dip in a water-based Gendine coating solution. Antimicrobial glove fingers as well as controls were tested using the International Standard for measurement of antibacterial activity on plastic surfaces (ISO 22196).

Briefly, films were secured over the opening of a 50 mL centrifuge tube to ensure the surface of the glove was flat and a target of 1×10⁵ CFU in 67 μL of bacterial inoculum was pipetted onto the surface of the film. A microscope coverslip (18 mm×18 mm) was placed on top of the bacterial inoculum and exposed for 1 minute. This was done to ensure a thin film of inoculum between the glove surface and coverslip. After exposure, all antimicrobial activity was halted by submersion in 10 mL of D/E Neutralizing Broth. Previous studies have shown that 10 mL of D/E Neutralizing Broth is sufficient for neutralization of the antimicrobial glove surface. Subsequent culture was recovered and serially diluted for quantitative culture. Resulting cultures were counted for growth. Gendine gloves were compared to controls to determine log reduction.

All antimicrobial Gendine gloves were tested against eight different multidrug resistant gram-positive, gram-negative, and yeast organisms. All organisms tested were MD Anderson clinical patient isolates routinely used for efficacy testing for device development; examples of these high level resistant organisms and their susceptibility profiles are found in Table 5.

TABLE 5
Resistance profile of organisms evaluated in ISO 22196.

| Organism | Resistance profile |
|---|---|
| Methicillin-resistant | Amox/Clav, Amp/Sulbactam, Cefazolin, |
| Staphylococcus aureus | Erythromycin, Oxacillin, Penicillin |
| (MRSA 4798) | |
| Vancomycin-resistant | Ampicilin, Gentamicin 500, Penicillin, |
| enterococci (VRE 3868) | Strep 2000, Vancomycin |
| MDR Pseudomonas | Aztreonam, Ceftizoxime, Ceftriaxone, |
| aeruginosa (PS 4689) | Ciprofloxacin, Imipenem, Levofloxacin, |
| | Meropenem, Norfloxacin, Ticar/Clav |
| MDA Acinetobacter | Amox/Clav, Cefepime, Ciprofloxacin, |
| baumanni | Imipenem, Moxifloxacin, Pipera/Tazo, |
| (AN 1021) | Ticar/Clav, Tigecycline, |
| | Tobramycin, Trimeth/Sulfa |
| Klebsiella pneumoniae | Ampicillin, Amp/Sulbactam, Aztreonam, |
| carbapenemase | Cefepime, Cefotaxime, Ceftazidime, |
| producing | Ceftriaxone, Ciprofloxacin, |
| (KPC 2855) | Levofloxacin, Moxifloxacin, Pipera/Tazo |

Results:

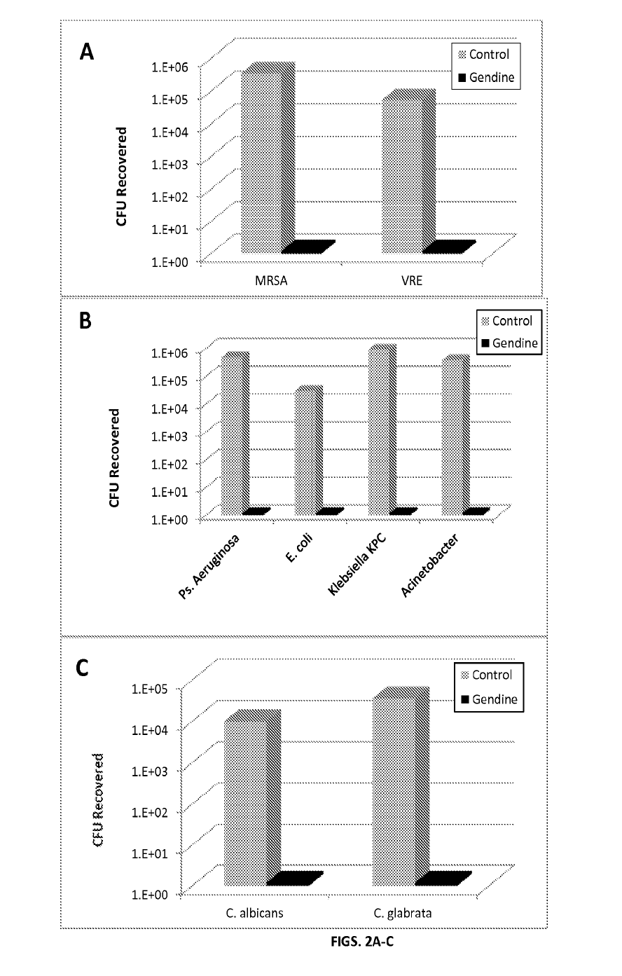

Gram positive, gram negative and yeast organisms recovered from control (uncoated) glove fingers were compared with organisms recovered from Gendine glove fingertips to determine efficacy and log reduction of Gendine coated nitrile gloves (FIGS. 2A-C, Table 6).

TABLE 6
Microbial organisms recovered from control
and Gendine-coated glove fingers.
Gram-Positive Organisms

| | MRSA | VRE |
|---|---|---|
| Control | 3.6 × 10⁵ | 5.4 × 10⁴ |
| Recovered | | |
| Gendine | 0 | 0 |
| (CFU/mL) | | |
| Log Reduction | 5.56 | 4.73 |

Gram-Negative Organisms

| | Ps. | | Klebsiella | |
|---|---|---|---|---|
| | aeruginosa | E. coli | KPC | Acinetobacter |
| Control | 4.9 × 10⁵ | 3.04 × 10⁴ | 9.2 × 10⁵ | 3.8 × 10⁵ |
| Recovered | | | | |
| Gendine | 0 | 0 | 0 | 0 |
| (CFU/mL) | | | | |
| Log Reduction | 5.69 | 5.48 | 5.96 | 5.58 |

Yeast

| | C. albicans | C. glabrata |
|---|---|---|
| Control | 1.0 × 10⁴ | 3.6 × 10⁴ |
| Recovered | | |
| Gendine | 0 | 0 |
| (CFU/mL) | | |
| Log Reduction | 4.0 | 4.56 |

Conclusions:

Using the ISO 22196 for evaluation of antimicrobial plastics (formerly JIS 2801), the inventors were able to demonstrate a high level of antimicrobial efficacy of Gendine-coated gloves for eight different multidrug resistant (pan resistant) organisms within a 1 minute exposure. Based on this coating solution, the inventors were able to demonstrate not only an endpoint of >4 log reduction but also complete eradication of organisms at 1 minute

### Example 3

### Coating Solutions with Added Silicone, Volatile fluids, and/or Non-Volatile Fluids to Reduce Tack

Gendine Glove Coating Solution:

100 ml of Gendine glove coating solution was made by preparing the following stock solutions: 10% partially hydrolyzed polyvinylacetate (88% hydrolyzed) in water (10 g in 100 ml); 20% porcine gelatin (200 bloom) in hot water (70° C.) (20 g in 100 ml); Chlorhexidine gluconate in water (20%) (20 g in 100 ml); Gentian Violet in water (0.1%) (100 mg in 100 ml).

100 ml Gendine Coating solution was made by mixing the stock solutions in the following ratios: 87.34 ml chlorhexidine gluconate+9.34 ml polyvinylacetate+3.12 ml gelatin+0.2 ml gentian violet. The solution was stirred well.

Coating of Nitrile Gloves Using Gendine Coating Solution:

Individual Nitrile Gloves were coated by spraying the surface with the Gendine glove coating solution using an atomizing spray bottle. The wetted gloves were dried using convective hot air. The gloves were tacky while wet and slightly tacky when fully dried.

Individual Nitrile Gloves were also coated by wetting the surface of a 4 inch long roller. The roller was rolled over both surfaces of the glove with gentle pressure to wet the glove. The wetted glove was then dried using convective hot air. Tack was assessed by compressing gloves together and then assessing mechanical resistance to being peeled apart. Gloves were tacky while wet but slightly tacky when fully dried.

Coating A Gloves:

a nontacky glove coating solution was prepared using cyclomethicone and PEG 8 dimethicone, PEG 33 dimethicone, and PEG 14 by the following method. To 30 grams of Gendine Coating solution was added 0.2 grams of SILSENSE Copolyol 1 (Lubrizol Corp), which contains a mixture of PEG 8 dimethicone, PEG 33 dimethicone, and PEG 14. 10 grams of d4 and d5 cyclomethicone was emulsified into the coating solution which was sprayed onto nitrile gloves. The gloves were dried by hot air convection. The gloves were not tacky during drying and were not tacky when dry.

Assessment of Antimicrobial Activity of Coating a Gloves:

The antimicrobial activity of the Coating A Gloves against methicillin-resistant Staphylococcus aureus (MRSA 4798) was performed using the method described above. Results are tabulated below for uncoated nitrile glove samples, gloves prepared with Gendine coating solution, and the d4 and d5 cyclomethicone modified coating (Coating A). Results are shown below in Table 7.

TABLE 7

| | MRSA colony counts |
|---|---|
| | (cfu) |
| Control Uncoated | |
| replicate 1 | ≧1.5E+06 |
| replicate 2 | ≧1.5E+06 |
| Gendine | |
| replicate 1 | 0 |
| replicate 2 | 0 |
| Gendine + d4 and d5 cyclomethicone Coating A) | |
| replicate 1 | 0 |
| replicate 2 | 0 |

Coating B Gloves:

Further modifications of the d4 and d5 cyclomethicone coating solution (coating A) were generated. More specifically, in addition the silicone copolyol additives in Coating A, 0.1 grams of nonvolatile dimethicone was added to the coating solution to produce Coating B. Gloves were then wetted and dried with Coating B. The dried glove had a wetter finish but was non tacky. 0.1 grams of fractionated coconut oil was substituted for the nonvolatile dimethicone with similar result.

Coating C Gloves:

The Coating C contains a gendine coating solution in cyclomethicone emulsion. More specifically, to 3 grams of Gendine coating solution was added 0.03 grams PEG 8 dimethicone. This was added to 10 grams of d4 and d5 cyclomethicone and emulsified. The water-in-silicone emulsion was immediately applied to gloves. A non-tacky coating formed during drying and following drying.

Coating D Gloves:

A nontacky glove coating using volatile dimethicone (0.65 centistoke) was prepared by the following method. 0.075 grams of PEG 8 dimethicone (Gransuf 62, Grant Industries Inc.) and 0.075 grams Cetyl PEG/PPG-10/1 dimethicone (Gransurf 90, Grant Industries Inc.) was added to 30 grams of the Gendine glove coating solution. Cetyl PEG/PPG-10/1 Dimethicone is the copolymer of Cetyl Dimethicone and an alkoxylated derivative of Dimethicone containing an average of 10 moles of ethylene oxide and 1 mole of propylene oxide. 3 grams of 0.65 centistoke dimethicone was then emulsified into the mixture. Gloves were coated both by rolling and spraying. The coated gloves were dried by hot air convection. The gloves were not tacky while drying and were non-tacky when dry.

Coating E Gloves:

A non-tacky glove coating using volatile dimethicone was prepared as follows. Nontacky gloves were prepared as described in Coating D gloves except 0.06 grams PEG 8 dimethicone and 0.12 grams of Cetyl PEG/PPG-10/1 dimethicone were added to 30 grams of Gendine coating solution. 6 grams of volatile dimethicone was emulsified and gloves were coated and dried using the same approach as for the Coating D gloves.

Coating F Gloves:

A non-tacky glove coating using volatile cyclomethicone was prepared as follows. Nontacky gloves were prepared as described in Coating D gloves except 0.06 grams PEG 8 dimethicone, 0.12 grams of Cetyl PEG/PPG-10/1 dimethicone and 0.1 grams of glycerol were added to 30 grams of Gendine coating solution. 10 grams of volatile cyclomethicone was added and vortexed to emulsify, and gloves were coated and dried using the same approach as for the Coating D gloves.

Coating G Gloves:

A non-tacky glove coating using volatile cyclomethicone was prepared as follows. Nontacky gloves were prepared as described in Coating D gloves except 0.06 grams PEG 8 dimethicone, 0.12 grams of Cetyl PEG/PPG-10/1 dimethicone and 0.1 grams of glycerol were added to 30 grams of Gendine coating solution. 15 grams of volatile cyclomethicone was added and vortexed to emulsify, and gloves were coated and dried using the same approach as for the Coating D gloves.

Coating H Gloves:

A non-tacky glove coating using volatile dimethicone was prepared as follows. Nontacky gloves were prepared as described in Coating D gloves except 0.06 grams PEG 8 dimethicone, 0.12 grams of Cetyl PEG/PPG-10/1 dimethicone and 0.1 grams of glycerol were added to 30 grams of Gendine coating solution. 15 grams of volatile dimethicone was added, vortexed to emulsify and gloves were coated and dried using the same approach as for the Coating D gloves.

Coating I Gloves:

A non-tacky glove coating using volatile cyclomethicone-dimethicone mixture was prepared as follows. Nontacky gloves were prepared as described in Coating D gloves except 0.06 grams PEG 8 dimethicone, 0.12 grams of Cetyl PEG/PPG-10/1 dimethicone and 0.1 grams of glycerol were added to 30 grams of Gendine coating solution. 7.5 grams of volatile dimethicone and 7.5 grams volatile cyclomethicone were added, vortexed to emulsify and gloves were coated and dried using the same approach as for the Coating D gloves.

Coating J Gloves:

A non-tacky glove coating using volatile cyclomethicone-dimethicone mixture was prepared as follows. Nontacky gloves were prepared as described in Coating D gloves except 0.035 grams PEG 8 dimethicone, 0.065 grams of Cetyl PEG/PPG-10/1 dimethicone and 0.12 grams of glycerol were added to 30 grams of Gendine coating solution. 2 grams of volatile dimethicone and 2 grams volatile cyclomethicone were added, vortexed to emulsify and gloves were coated and dried using the same approach as for the Coating D gloves.

Antimicrobial Efficacy of Coating D, E and J Gloves.

Coating D, E and J Gloves were tested for antimicrobial efficacy using the microbiological challenge method described above and used to test Coating A Gloves. Results are shown below in Table 8.

TABLE 8

| | | Pseudomonas | Candida |
|---|---|---|---|
| Sample | MRSA | aeruginosa | albicans |
| Control untreated | | | |
| Replicate 1 | >1.50 × 10⁶ | >1.50 × 10⁶ | >1.50 × 10⁶ |
| Replicate 2 | >1.50 × 10⁶ | >1.50 × 10⁶ | >1.50 × 10⁶ |
| Gendine | | | |
| Replicate 1 | 0 | 0 | 0 |
| Replicate 2 | 0 | 0 | 0 |
| Coating D | | | |
| Replicate 1 | 0 | 0 | 0 |
| Replicate 2 | 0 | 0 | 0 |
| Coating E | | | |
| Replicate 1 | 0 | 0 | 0 |
| Replicate 2 | 0 | 0 | 0 |
| Coating J | | | |
| Replicate 1 | 0 | 0 | 0 |
| Replicate 2 | 0 | 0 | 0 |

Organism and ATCC #: Methicillin-Resistant Staphylococcus aureus - MRSA 4798; Pseudomonas aeruginosa - PS 4689; Candida albicans - CA 009-3072.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,364,918
U.S. Pat. No. 6,120,904
U.S. Pat. No. 4,729,914
U.S. Pat. No. 7,553,511
U.S. Pat. No. 6,630,152
U.S. Pat. No. 8,137,735
U.S. Pat. No. 5,357,636
U.S. Pat. No. 5,438,709
U.S. Pat. No. 4,853,978
U.S. Pat. No. 6,630,152
U.S. Patent Application 2011/0099688
U.S. Patent Application 2008/0081020,
U.S. Patent Application 2010/0233223,
U.S. Patent Application 2008/0183152
U.S. Patent Application 2004/0126604
U.S. Patent Application 2004/0241201
K. Allmear, A. Hult, B. Ramb. Surface modification of polymers. I. Vapour phase photografting with acrylic acid. Journal of Polymer Science Part A: Polymer Chemistry 1988; 26(8):2099-2111.
Y. Uyama, K. Kato, Y. Ikada. Surface modification of polymers by grafting. Advances in Polymer Science 1998; 137:1-39.
D. Hegemann, H. Brunner, C. Oehr. Plasma treatment of polymers for surface and adhesion improvement. Nuclear Instruments and Methods in Physical Research Section B:

Beam Interaction with Materials and Atoms 2003; 208:281-286.
C. Weikart, H. Yasuda. Modification, degradation and stability of polymeric surfaces treated with reactive plasmas. Journal of Polymer Science Part A: Polymer Chemistry 2000; 38(17):3028-3042.
L. Ferreira, A. Falcao, M. Gil. New LDPE copolymeric films with enhanced hydrophilic properties prepared by gamma irradiation. Material Science Forum 2006; 514-516:1034-1038.
Z. Ma, C Gao, J Yuan, Y Gong, J Shen. Surface modification of poly-l-lactide by photografting of hydrophilic polymers towards improving its hydrophilicity. Journal of Applied Polymer Science 2002; 85(10): 2163-2171.
J. Bico, U. Thiele, D. Quere. Wetting of textured surfaces. Colloids and Surfaces A: Physicochemical and Engineering Aspects. 2002; 206(1-3): 41-46.
S. Fotiadou, K Chrissopoulou, B. Frick, S. Anastasiadis. Structure and dynamics of polymer chains in hydrophilic nanocomposites. Journal of Polymer Science Part B: Polymer Physics 2010; 48(14): 1658-1667.
B. Coupe, M. Evangelista, R. Yueng, W. Chen. Surface modification of poly(tetrafluoroethylene-co-hexafluoropropylene) by adsorption of functional polymers. Langmuir 2001; 17(6): 1956-1960.

1. A medical device comprising a polymer-based surface, wherein an antimicrobial composition is present on at least a portion of the surface, and wherein the antimicrobial composition comprises:
(a) chlorhexidine and a partially hydrolyzed polyvinyl acetate copolymer with a vinyl acetate content of from about 5% to about 95%; or
(b) an aqueous or multiphase solution comprising a biguanide and a silicone; wherein if the silicone is a volatile silicone, then at least 0.01% of the volatile silicone can volatilize from the polymer based surface within 48 hours at about 50° C.; and wherein if the silicone is a nonvolatile silicone, then the ratio of biguanide/nonvolatile silicone is greater than 12.

2. The medical device of claim 1, wherein the antimicrobial composition comprises chlorhexidine and a partially hydrolyzed polyvinyl acetate copolymer with a vinyl acetate content of from about 5% to about 95%.

3. The medical device of claims 1-2, wherein the partially hydrolyzed polyvinyl acetate copolymer is a partially hydrolyzed vinyl alcohol vinyl acetate copolymer.

4. The medical device of claims 1-3, wherein the chlorhexidine comprises greater than about 85% (w/w) of the antimicrobial composition.

5. The medical device of claim 4, wherein the chlorhexidine comprises greater than about 90% (w/w) of the antimicrobial composition.

6. The medical device of claims 1-4, wherein the chlorhexidine is chlorhexidine gluconate.

7. The medical device claims 1-6, wherein the antimicrobial composition further comprises gentian violet or brilliant green.

8. The medical device of claims 1-6, wherein the partially hydrolyzed polyvinyl acetate copolymer comprises from about 10% to about 50% vinyl acetate.

9. The medical device of claim 8, wherein the partially hydrolyzed polyvinyl acetate copolymer comprises from about 10% to about 25% vinyl acetate.

10. The medical device of claims 1-9, wherein the partially hydrolyzed polyvinyl acetate copolymer comprises less than about 15% of the composition.

11. The medical device of claim 10, wherein the partially hydrolyzed polyvinyl acetate copolymer comprises from about 3% to about 5% of the composition.

12. The medical device of claims 1-11, wherein the antimicrobial composition further comprises a gel-forming protein or gel-forming carbohydrate.

13. The medical device of claim 12, wherein the antimicrobial composition further comprises a gel-forming protein, wherein the gel-forming protein is a gelatin, a collagen, a starch, a chitosan, an alginate, or a gum.

14. The medical device of claim 13, wherein the gel-forming protein is gelatin.

15. The medical device of claim 12, wherein the antimicrobial composition further comprises a gel-forming carbohydrate, wherein the gel-forming carbohydrate is a starch, chitosan, or alginate.

16. The medical device of claims 1-15, wherein the gel-forming protein or gel-forming carbohydrate comprises from about 0.1% to about 5% of the composition.

17. The medical device of claim 16, wherein the gel-forming protein or gel-forming carbohydrate comprises from about 1% to about 3% of the composition.

18. The medical device of claims 1-17, wherein the medical device is a glove.

19. The medical device of claim 18, wherein the glove is a latex glove, a polyvinyl chloride glove, a nitrile glove, or a glove made of a laminate or blend of latex, polyvinylchloride, or nitrile.

20. The medical device of claims 1-19, wherein the medical device is sterile.

21. The medical device of claims 1-20, wherein the antimicrobial composition comprises an aqueous solution, suspension, or emulsion.

22. The medical device of claims 1-21, wherein the antimicrobial composition comprises an aqueous or multiphase solution, suspension, or emulsion comprising a biguanide and a silicone; wherein if the silicone is a volatile silicone, then at least 0.01% of the volatile silicone can volatilize from the polymer based surface within 48 hours at about 50° C.; and wherein if the silicone is a nonvolatile silicone, then the ratio of biguanide/nonvolatile silicone is greater than 12.

23. The medical device of claims 1-22, wherein the antimicrobial composition comprises an aqueous or multiphase solution comprising a biguanide and a volatile silicone, wherein at least 0.01% of the volatile silicone can volatilize from the polymer based surface within 48 hours at about 50° C.

24. The medical device of claims 1-23 wherein the antimicrobial composition comprises a multiphase solution comprising a plurality of volatile phases that can concurrently volatilize from the polymer based surface within 48 hours at about 50° C.

25. The medical device of claims 1-24, wherein the antimicrobial composition contains a volatile silicone and a humectant.

26. The medical device of claims 1-25, wherein the antimicrobial composition comprises a solution or multiphase solution comprising a biguanide and a nonvolatile silicone, wherein the ratio of biguanide/nonvolatile silicone is greater than 12.

27. The medical device of claims 1-26, wherein the biguanide concentration is greater than about 10%.

28. The medical device of claims 1-27, wherein the antimicrobial composition further comprises a partially hydrolyzed polyvinylacetate.

29. The medical device of claim 28, wherein the partially hydrolyzed polyvinyl acetate copolymer comprises from about 10% to about 50% vinyl acetate.

30. The medical device of claim 29, wherein the partially hydrolyzed polyvinyl acetate copolymer comprises from about 10% to about 25% vinyl acetate.

31. The medical device of claim 28, wherein the partially hydrolyzed polyvinyl acetate copolymer comprises less than about 15% of the composition.

32. The medical device of claim 31, wherein the partially hydrolyzed polyvinyl acetate copolymer comprises from about 3% to about 5% of the composition.

33. The medical device of claims 1-32, wherein the biguanide is chlorhexidine, chlorhexidine gluconate, chlorhexidine salt, alexidine, octenidine, or polyhexamethyl biguanide.

34. The medical device of claims 1-33, wherein the volatile silicone is a cyclomethicone.

35. The medical device of claim 34, wherein the cyclomethicone is a cyclotrisiloxane, cyclotetrasiloxane, cyclopentasiloxane, cyclohexasiloxane, or mixtures thereof.

36. The medical device of claims 1-33, wherein the volatile silicone is a dimethicone.

37. The medical device of claim 36, wherein the dimethicone contains disiloxane, trisiloxane, tetrasiloxane, pentasiloxane, hexasiloxane, a polysiloxane or mixtures thereof.

38. The medical device of claims 1-37, wherein the volatile silicone comprises a mixture of a dimethicone and a cyclomethicone.

39. The medical device of claims 1-38, wherein the nonvolatile silicone is a longer chain dimethicone having a viscosity of greater than 50 centistokes, a dimethicone copolymer, a dimethicone copolyol, a dimethicone crosspolymer, a silicone glycol, a cetaryl dimethicone copolymer, a dimethiconol fluid, a PEG-dimethicone, a PPG-dimethicone, a PEG/PPG-dimethicone, a cetyl dimethicone, an alkyldimethicone, an alkyl silicone, a polyether silicone, a hydroxyl silicone, a polyether dimethicone, a silicone surfactant or emulsifier, an aminodimethicone, an amodimethicone, an alkoxylated dimethicone, a cyclic siloxane, or a siloxane copolymer.

40. The medical device of claim 39, wherein the nonvolatile silicone is a cyclic, grafted, or crosslinked siloxane.

41. The medical device of claims 1-40, wherein the antimicrobial composition further comprises a hydrophobic oil or humectant, wherein the oil or humectant is pharmacologically acceptable for topical application to the skin.

42. The medical device of claim 41, wherein the oil is coconut oil, an unsaturated alkanoic acid, an aloe oil, a tocopherol, a tocotrienol, or a dimethicone.

43. The medical device of claim 41, wherein the humectant is glycerol, propylene glycol, polyethylene glycol, a polyethelene glycol polypropylene glycol copolymer, a polysorbate, a monogylyceride, diglyceride, triglyceride, fatty acid ether, glycerol or glycol conjugate.

44. The medical device of claims 1-42, wherein the antimicrobial composition comprises cyclomethicone and PEG 8 dimethicone, PEG 33 dimethicone, and PEG 14.

45. The medical device of claims 1-44, wherein the antimicrobial composition further comprises nonvolatile dimethicone.

46. The medical device of claims 1-45, wherein the antimicrobial composition comprises gendine solution in a cyclomethicone emulsion.

47. The medical device of claims 1-46, wherein the cyclomethicone emulsion comprises PEG 8 dimethicone and a d4 or d5 cyclomethicone.

48. The medical device of claims 1-47, wherein the antimicrobial composition comprises PEG 8 dimethicone and Cetyl PEG/PPG-10/1 dimethicone.

49. The medical device of any one of claims 1-17 and 19-48, wherein the medical device is a glove.

50. The medical device of claim 49, wherein the glove is a latex glove, a nitrile glove, a vinyl polymer glove, or a vinylidene polymer glove.

51. The method of any one of claims 1-17 and 19-48, wherein the medical device is a respiratory mask, a hair net or head cover, an exam table cover, an ear plug, a stethoscope cover, a footcover, a seatcover, a drape, a towel, a drain, a bag or pouch, a holder, a film, a tube, a gauze, a sponge, a mesh, a filter, a pad, a clip, a bandage, a drape, or an article of clothing.

52. A method of imparting antimicrobial activity to a polymer-based surface, comprising contacting at least a portion of a polymer-based surface with the antimicrobial solution, and substantially drying the contacted surface; wherein the antimicrobial solution comprises:
(a) chlorhexidine and a partially hydrolyzed polyvinyl acetate copolymer with a vinyl acetate content of greater than about 5%;
(b) an aqueous or multiphase solution comprising a biguanide and a silicone; wherein if the silicone is a volatile silicone, then at least 0.01% of the volatile silicone can volatilize from the polymer based surface within 48 hr at about 50° C.; and wherein if the silicone is a nonvolatile silicone, then the ratio of biguanide/nonvolatile silicone is greater than 12; or
(c) applying an aqueous or multiphase biguanide solution to the surface, and subsequently applying a silicone solution to the surface, wherein if the silicone is a volatile silicone, then at least 0.01% of the volatile silicone can volatilize from the polymer based surface within 48 hr at about 50° C.; and wherein if the silicone is a nonvolatile silicone, then the ratio of biguanide/nonvolatile silicone is greater than 12.

53. The method of claim 52, wherein the partially hydrolyzed polyvinyl acetate copolymer is a partially hydrolyzed vinyl alcohol vinyl acetate copolymer.

54. The method of claims 52-53, wherein the solution further comprises a gel-forming protein or gel-forming carbohydrate.

55. The method of claims 52-54, wherein the solution comprises gelatin.

56. The method of claims 52-55, wherein the solution comprises greater than 85% chlorhexidine.

57. The method of claim 56, wherein the solution comprises greater than 90% chlorhexidine.

58. The method of claim 57, wherein the solution comprises about 95% chlorhexidine.

59. The method of claims 52-58, wherein the solution comprises about 95% chlorhexidine, about 3% partially hydrolyzed vinyl alcohol vinyl acetate copolymer, and about 2% gelatin.

60. The method of claims 52-59, wherein the surface comprises a nitrile, a latex, a polyvinyl chloride, a polyester, a polyamide, a polyurethane, a cellulosic, or an olephin.

61. The method of claims 52-60, wherein the surface is a vinyl-based surface.

62. The method of claims 52-61, wherein the surface is comprised on a medical device.

63. The method of claim 62, wherein the medical device is a respiratory mask, a hair net or head cover, an exam table cover, an ear plug, a stethoscope cover, a footcover, a seatcover, a drape, a towel, a drain, a bag or pouch, a holder, a film, a tube, a gauze, a sponge, a mesh, a filter, a pad, a clip, a bandage, a drape, or an article of clothing.

64. The method of claims 61-63, wherein the antimicrobial composition is coated on a portion of the vinyl-based surface at a concentration effective to kill a bacterium or a fungus.

65. The method of any one of claims 52-62 and 64, wherein the surface is comprised on a glove.

66. The method of claim 65, wherein the glove is a latex glove, a polyvinyl chloride glove, or a nitrile glove.

67. The method of claims 52-66, wherein the polymer-based surface is contacted with a reactive gas, a gas-plasma, a reactive liquid, a interpenetrating network, or a hydrophilic coating, or is exposed to radiation treatment.

68. The method of claims 52-67, wherein said contacting comprises dipping or spraying.

69. The method of claims 52-68, wherein the method further comprises using a roller process to apply the antimicrobial solution to the surface and dry the surface.

70. The method of claims 52-69, wherein a heat tumble dryer is used to dry the antimicrobial solution on the surface.

71. The method of claims 52-70, wherein the surface is comprised on a glove.

72. The method of claim 71, wherein the glove is a latex glove, a polyvinyl chloride glove, or a nitrile glove.
